(19) United States
(12) Patent Application Publication
(10) Pub. No.: US 2025/0281278 A1
(43) Pub. Date: Sep. 11, 2025

(54) CORNEAL IMPLANT

(71) Applicants: AJL OPHTHALMIC, S.A., Miñano (ES); FUNDACIÓN DE INVESTIGACIÓN OFTALMOLÓGICA FERNÁNDEZ-VEGA, Oviedo (ES)

(72) Inventors: Eva Larra Mateos, Miñano (ES); Jesús Perez Gracia, Miñano (ES); Fernando José Alfonso Sánchez, Urb. La Fresneda-Siero (ES)

(21) Appl. No.: 19/219,650

(22) Filed: May 27, 2025

Related U.S. Application Data
(63) Continuation of application No. 17/424,612, filed on Jul. 21, 2021.

(30) Foreign Application Priority Data
Jan. 22, 2019 (EP) 19382042.0

(51) Int. Cl. A61F 2/14; C08L 33/12

(52) U.S. Cl. CPC A61F 2/142; C08L 33/12; A61F 2230/0006; A61F 2250/0091

(57) ABSTRACT

The present disclosure relates to a corneal implant, particularly the implant is useful for a corneal transplant. The implant avoids direct contact between the patient tissue and a donated cornea. The implant comprises: a disc in the centre of the implant, a ring in a superior plane parallel to the disc, and a radial connecting element between the disc and the ring wherein the implant is configured to perform the function of a mechanical barrier between the donor's cornea and the host descemet endothelium.

### CROSS REFERENCE TO RELATED APPLICATIONS

The present application is a continuation of U.S. patent application Ser. No. 17/424,612, filed Jul. 21, 2021, which is a § 371 national stage of International Application PCT/EP2020/051421, filed Jan. 21, 2020, which claims priority benefit of European Pat. Application Ser. No. 19382042.0, filed Jan. 22, 2019, all of which are hereby incorporated herein by reference in their entireties.

### FIELD

The present invention relates to a corneal implant, particularly the implant is useful for a corneal transplant, the implant avoids direct contact between the patient tissue and a donated cornea.

### BACKGROUND ART

Diseases affecting the cornea are a major cause of global vision loss. Some pathology may lead to corneal dysfunction necessitating corneal transplantation.

There are three basic types of corneal transplantations. Full thickness transplants, the anterior lamellar transplant, if we replace the epithelium and the stroma, and the posterior lamellar transplant, if we replace the endothelium.

Complications of a corneal transplant can be significant and can include cornea graft rejection and eye infection.

Rejection of the donor tissue is the most serious complication after a corneal transplant.

The patent document U.S. Pat. No. 8,388,608 B1 describes the developing of a new method for attaching the donor transplant cornea or artificial cornea without the use of sutures by means of a special implant. This method tries to overcome the problems associated with the sutures based on precise cuts in the cornea.

While full thickness corneal transplants have not changed much over the past century, lamellar corneal transplant techniques have evolved rapidly.

In some cases when there was a bad prognosis for a full thickness transplant, the possibility of associating a prosthesis to the donor cornea with the intention of improving this prognosis was proposed.

One example is the artificial cornea sold under the trademark Boston Keratoprothesis™. It was originally made of polymethylmethacrylate (PMMA) in a collar button design, to be implanted into a corneal graft carrier and then transplanted to the patient's cornea. The risks associated are: postoperative infections and again the main problem is the rejection of the donor tissues. Also ocular hypertension, and mechanical weakness of the cornea in cases of trauma are problems arising from perforation of the cornea.

In connection with this techniques of lamellar transplant, the patent application publication EP1428470A2 describes an apparatus for determining and ablating a corneal tissue volume of a receiving cornea for corneal lamellar grafting transplantation.

The prognosis of corneal transplants with the introduction of these lamellar techniques has improved significantly, so today they are considered to be the first choice techniques as long as the criteria for their surgical indication are met.

Thus, from what is known in the art, it is derived that there is still great interest to develop an implant transplant to improve some of the problems associated with the actual techniques.

### SUMMARY OF THE INVENTION

The cornea is the transparent layer forming the front of the eye. The tissues of the cornea are arranged in three basic layers: epithelium-bowman, stroma and descemet-endothelium.

The epithelium-bowman is the cornea's outermost layer. The second layer is the stroma, which is the thickest layer of the cornea. The descemet-endothelium is the innermost layer of the cornea.

The corneal transplantation is a widely practiced surgical procedure. There are two main types of cornea transplant: full thickness cornea, and layer cornea transplant, wherein not all the cornea is eliminated, called lamellar transplantation.

The present implant is useful in the transplant wherein not all of the cornea's layers are eliminated. The implant of the present invention is a mechanical barrier between the donor's cornea and the host descemet-endothelium, i.e. the patient's descemet-endothelium.

When the two superficial layers of the cornea, epithelium and stroma, are eliminated (as when it is carried out in a previous lamellar transplant) a donor cornea replaced them, with its total thickness, that is, with its three layers, epithelium-bowman, stroma and descemet-endothelium. In the space formed between the donor cornea and the deep layer of the receiving cornea, the implant of the invention is inserted in order to keep both corneas separated.

Therefore, an aspect of the present invention relates to an implant comprising: a planar element in the centre of the implant, a ring in a superior plane parallel to the planar element, and a radial connecting element between the planar element and the ring.

The implant of the invention shows many advantages, the risk of the rejection is minimized because there is not a contact between the donor and the host tissues.

The method to implant the implant of the invention comprises the following steps:
a) eliminating the epithelium-bowman and stroma layers of the host;
b) positioning the implant defined above on the host's descemet-endothelium with the planar element by direct contact with the descemet-endothelium and the ring in a superior plane parallel to the planar element;
c) positioning the donor's cornea in the ring of the implant;
d) suturing the donor's and the host's corneas.

The donor's tissue and the patient (host) tissue are separated; there is a chamber between them, originating from the implant. This chamber is similar to the anterior chamber of the ocular globe (we name it, pseudochamber).

In the method described above the patient cornea is not perforated. Perforation is one of the steps that causes the most serious problems in a cornea's transplantation.

After placement of the implant in its correct position, over the descemet-endothelium and the positioning of the donor's cornea on top of the implant, on the ring, the donor's cornea is sutured to the peripheral corneal part of the patient, to ensure its union and favor proper healing.

### BRIEF DESCRIPTION OF THE DRAWINGS

FIG. 1 shows a perspective view of the implant.

### DETAILED DESCRIPTION OF THE INVENTION

As mentioned above, an aspect of the present invention relates to an implant comprising:
a planar element in the centre of the implant, a ring in a superior plane parallel to the planar element, and a radial connecting element between the planar element and the ring.

The connecting element between the planar element in the centre of the implant and the ring is situated in the radial direction, from the centre of the implant to the ring.

In a preferred embodiment the planar element is a disc.

The term disc means a flat, circular element.

In a particular embodiment the diameter of the ring is greater than the diameter of the disc.

In a particular embodiment the diameter of the ring is comprised between 7.5 mm and 9.0 mm. More particularly, the diameter of the ring is comprised between 8.0 mm and 8.5 mm.

In a particular embodiment the connecting element is at least a rib interconnecting the planar element and the ring in the superior plane. More particularly, the at least a rib are four ribs separate from one another in an azimuthal direction by an angle of 90°.

Particularly the implant is made of poly (methyl methacrylate) (PMMA).

The poly (methyl methacrylate) is a hard, rigid, and glassy, but brittle material. Also, it is an inert material usually used in intraocular lenses and hard contact lenses. PMMA has also been used in intraocular implants and as intrastromal corneal rings due to its mechanical and optical properties.

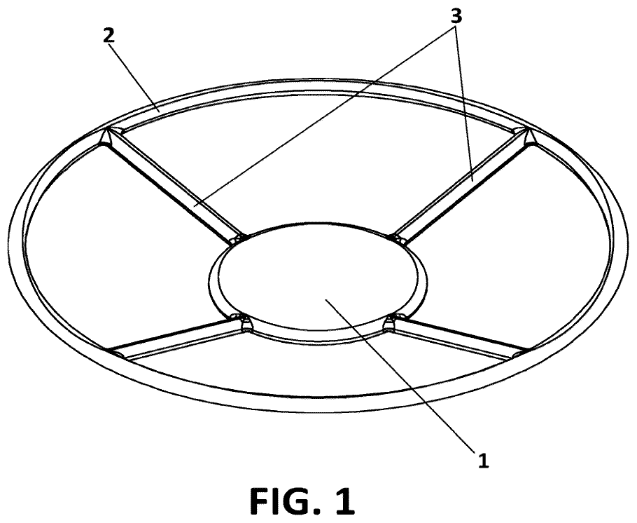

FIG. 1 shows a perspective of the implant. In this particularly embodiment the implant shows a central disc (1) in the centre of the implant, a ring (2) in a superior parallel plane and four ribs (3) connecting the two elements, disc (1) and ring (2).

1. An implant comprising:
a disc in the centre of the implant;
a ring in a superior plane parallel to the disc; and
a radial connecting element between the disc and the ring;
wherein the implant is configured to perform the function of a mechanical barrier between a donor's cornea and a host's Descemet endothelium.

2. The implant according to claim 1 wherein the diameter of the ring is greater than the diameter of the disc.

3. The implant according to claim 1 wherein the diameter of the ring is between 7.5 mm and 9.0 mm.

4. The implant according to claim 1 wherein the diameter of the ring is between 8.0 mm and 8.5 mm.

5. The implant according to claim 1 wherein the radial connecting element is at least a rib interconnecting the disc and the ring in the superior plane.

6. The implant according to claim 5 wherein the at least a rib are four ribs separated from one another in an azimuthal direction by an angle of 90°.

7. The implant according to claim 2 wherein the radial connecting element is at least a rib interconnecting the disc and the ring in the superior plane.

8. The implant according to claim 3 wherein the radial connecting element is at least a rib interconnecting the disc and the ring in the superior plane.

9. The implant according to claim 4 wherein the radial connecting element is at least a rib interconnecting the disc and the ring in the superior plane.

10. A method of implanting a corneal implant, the method comprising:
eliminating the epithelium-bowman and stroma layers of a host's cornea;
positioning the corneal implant on the host's descemet-endothelium, the corneal implant having a planar element placed into direct contact with the descemet-endothelium and a ring in a superior plane parallel to the planar element;
positioning a donor's cornea in the ring of the implant; and
suturing the donor's and the host's corneas;
wherein the host's cornea is not perforated during said positioning and suturing steps.

11. The method of claim 10, wherein said suturing the donor's cornea is performed after said positioning the corneal implant on the host's descemet-endothelium and said positioning the donor's cornea in the ring of the implant, wherein said suturing the donor's cornea comprises suturing the donor's cornea to the peripheral corneal part of the host.
